(19) United States
(12) Patent Application Publication
(10) Pub. No.: US 2007/0023051 A1
(43) Pub. Date: Feb. 1, 2007

(54) Inflatable airways with pressure monitoring devices

(76) Inventor: Daniel J. Cook, St. Louis, MO (US)

Correspondence Address: LEWIS, RICE & FINGERSH, LC;ATTN: BOX IP DEPT., 500 NORTH BROADWAY, SUITE 2000, ST LOUIS, MO, 63102, US

(21) Appl. No.: 11/194,855

(22) Filed: Jul. 30, 2005

(51) Int. Cl. A61M 16/00

(52) U.S. Cl. 128/207.15

(57) ABSTRACT

An artificial inflatable airway device is described that includes at least one inflatable sealing portion and a pressure monitoring means, the sealing portion and the pressuring monitoring means being integrally and operably attached, adapted to measure at least one pressure within the sealing portion of the inflatable airway.

### BACKGROUND OF THE INVENTION

This invention relates to artificial airway devices, and more specifically to airway devices that include pressure monitoring systems.

High pressure within the sealing portion of airway devices, such as endotracheal tubes, tracheal tubes, esophogeal obterator airways, supraglottic laryngeal airways, laryngeal airways and pharyngeal airways can lead to specific tissue, vascular, or nerve injuries to the structures in the vicinity of the area that the devices are designed to seal. Due to these risks, the U.S. Food and Drug Administration encourages the manufacturers of these airway devices to disclose the maximum pressures at which the devices can be used safely. Ironically, however, no integral continuous or intermittent devices exist to monitor the pressures that exist within the devices during use. Accordingly, it is usually unknown whether the devices are actually at a safe pressure during use.

### SUMMARY OF THE INVENTION

Briefly, in a first aspect of the invention, an inflatable airway device is provided that comprises at least one inflatable sealing portion and a pressure monitoring means integrally and operably connected to the sealing portion, the pressure monitoring means adapted to measure at least one pressure within the sealing portion of the inflatable airway;

In another aspect of the invention, an inflatable airway device is provided that comprises at least one laryngeal airway comprising an inflatable sealing portion and at least one balloon assembly and a pressure monitoring means integrally attached to the at least one laryngeal airway, the pressure monitoring means within the at least one balloon assembly and comprising a gauge adapted to continuously measure at least one pressure level within the sealing portion of the laryngeal airway, wherein the pressure monitoring means is adapted to continuously report the pressure within the sealing portion of the laryngeal airway device.

In a further aspect of the invention, an inflatable airway device is provided that comprises at least one laryngeal airway comprising an inflatable sealing portion and at least one balloon assembly and a pressure monitoring means integrally attached to the at least one laryngeal airway, the pressure monitoring means within the at least one balloon assembly and comprising a gauge adapted to intermittently measure at least one pressure level within the sealing portion of the laryngeal airway, wherein the pressure monitoring means is adapted to intermittently report the pressure within the sealing portion of the laryngeal airway device.

### BRIEF DESCRIPTION OF THE DRAWINGS

In the drawings, FIG. 1 illustrates a pressure monitoring means in accordance with the invention comprising a valve and a pressure gauge that includes a movable membrane that may act as a pressure indicator;

FIG. 2 is an illustration of the gauge illustrated in FIG. 1 in a low pressure A and high pressure B position;

FIG. 3 is an exploded view of the gauge illustrated in FIG. 1;

FIG. 4 is an alternative embodiment of the invention comprising a pressure gauge that employs a spring; and

FIGS. 5-7 illustrate another alternative embodiment of the invention comprising an accordion-like membrane as an indicator.

### DESCRIPTION OF THE PREFERRED EMBODIMENTS

New and useful inflatable artificial airway devices have been discovered that comprise at least one inflatable sealing portion and a pressure monitoring means adapted to measure at least one pressure within the sealing portion of the inflatable airway. The airway device may be any airway device, including but not limited to those encouraged to disclose the maximum pressure levels for safe use to the U.S. Food and Drug Administration. Generally, the airway devices include at least one pressurized portion within the inflatable sealing portion. The airway devices include, but are not limited to endotracheal tubes, tracheal tubes, supraglottic laryngeal airways, laryngeal airways, esophogeal obterator airways and pharyngeal airways.

The pressure monitoring means 20 of the invention may be any device capable of measuring and/or monitoring at least one pressure within the sealing portion of the inflatable airway. The pressure monitoring means 20 measures the pressure within the sealing portion 10 of the airway device and is integral with the sealing portion 10 of the airway device. The pressure monitoring means and the sealing portion are in integral and operable attachment or connection to the sealing portion 10 of the inflatable airway. As used herein “operable attachment” or “connection” is any attachment or connection that facilitates the monitoring of the pressure within the sealing portion of the airway device. As used herein “integral” means that the pressure monitoring means and the sealing portion 10 of the inflatable airway, once constructed, are not separable while maintaining the structure and function of the airway and/or monitoring means. The pressure monitoring means 20 is not designed to be removed from the sealing portion 10 of the inflatable airway after use. The pressure monitoring means 20 is attached to the sealing portion 10 of the inflatable airway in such a way that removal of the pressure monitoring means 20 may disable the airway and/or disrupt the ability of the monitoring means 20 to continuously or intermittently monitor or report pressures. As used herein the phrase “sealing portion” refers to any inflatable or pressurized portion of the airway including but not limited to the sealing balloon, and/or assembly, pilot balloon assembly, inflation/deflation apparatus, inflation/deflation valve, or any device connected to the pressurized portion.

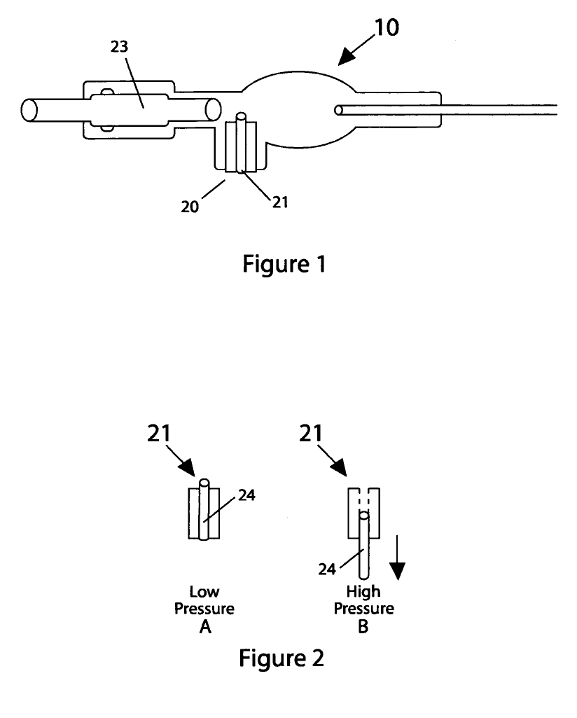

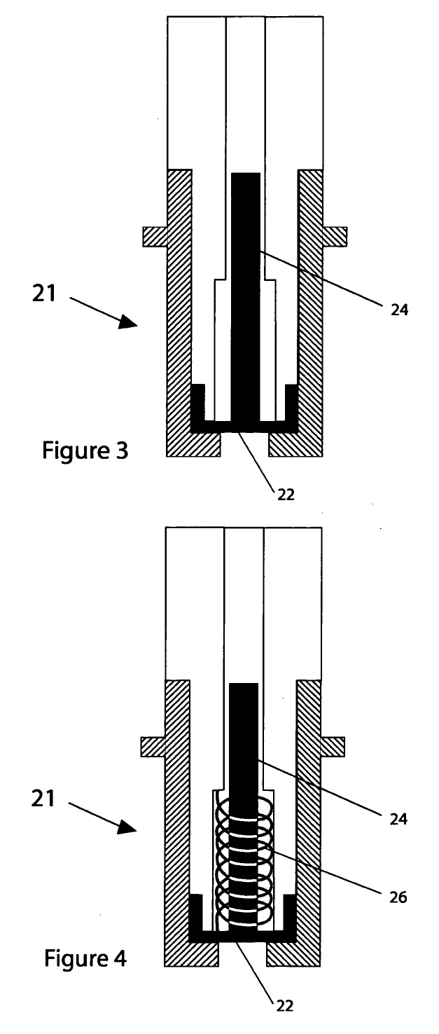

With reference to FIGS. 1-3, and in specific embodiments of the invention, the pressure monitoring means 20 is within the sealing portion 10 of the inflatable airway device. In one specific embodiment of the invention, the pressure monitoring means 20 comprises a pressure gauge 21 that is adapted to report the pressure level inside the airway device to the user. This embodiment also comprises a valve 23. As illustrated in FIG. 2, to measure the pressure within the sealing portion 10 of the airway device, the pressure gauge 21 comprises a movable member 24 that reacts to the level of pressure within the sealing portion 10 of the airway device. As the sealing portion 10 of the airway portion approaches a maximum or other predetermined pressure level the movable member moves from a closed, low pressure position A to a high pressure, open position B.

In a specific embodiment of the invention, the inflatable sealing portion 10 is a balloon assembly or pilot balloon assembly. In the embodiment shown in FIG. 1-3, the pressure monitoring means 20 is a pressure gauge 21 that lies within the pilot balloon assembly. In this embodiment, the pressure monitoring means 20 comprises a gauge 21 with a movable membrane 22, which may act as an indicator 24, or in conjunction with an indicator 24. It should be noted that this is one embodiment of the invention. Any artificial airway device may be used in accordance with the invention and any pressure monitoring means 20 may be employed without departing from the spirit and scope of the invention. It should also be noted that the indicator 24 may be any indicator including, but not limited to an inverted column that pops out when the pressure inside the balloon reaches a predetermined level, as shown in FIGS. 1-3.

In another embodiment of the invention illustrated in FIG. 4, the pressure monitoring means 20 comprises a gauge 21 with a membrane 22 that may act as an indicator 24 or in conjunction with an indicator 24 and a spring 26 which aids in tensioning the membrane 22 and in retracting the indicator 24 when the pressure falls below the prescribed level. Any artificial airway device may be used in accordance with the invention and any pressure monitoring means 20 may be employed without departing from the spirit and scope of the invention.

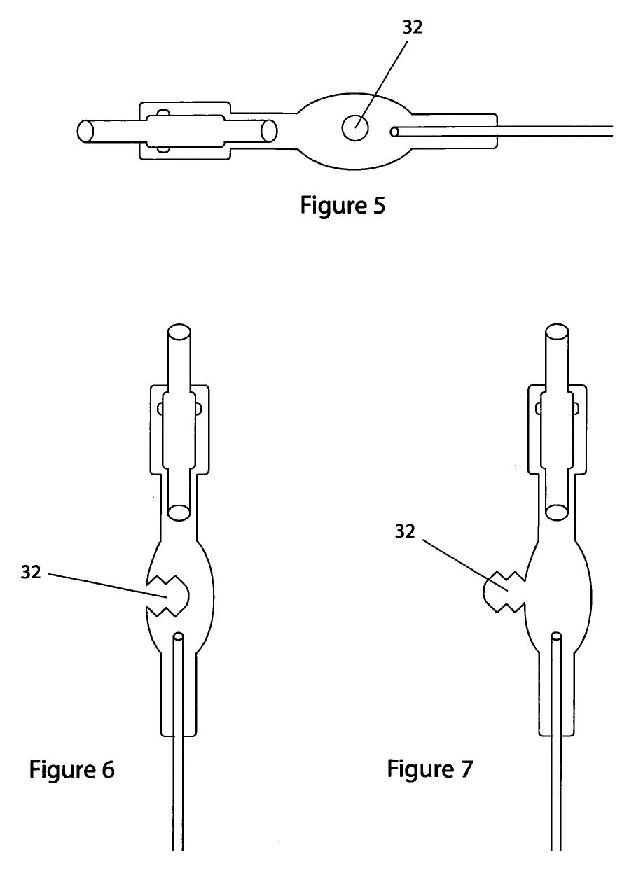

In yet another specific embodiment of the invention, the pressure monitoring means 20 is built into the pilot balloon and comprises an inverted column, as illustrated in FIGS. 5-7, or an accordion-like membrane 32 that pops out when it reaches a certain, predetermined pressure level, as illustrated in FIGS. 5-7. The pressure monitoring means 20 would generally be in-molded into, and a part of, a portion of the pressurized portion 10, more specifically into the pilot balloon. When the pressure is released, the column may need to be manually pushed back, or may be self-retracting. Again, it should be noted that any pressure monitoring means 20 may be employed in accordance with the invention.

The pressure monitoring means is adapted to measure the level of pressure within the sealing portion 10 of the inflatable airway device. The pressure monitoring means may also be adapted to report the level of pressure within the sealing portion 10 of the inflatable airway device. Other devices, however, may be employed to report information relating to the pressure level inside the sealing portion 10 to the user. As used herein, reporting the level of pressure includes reporting a specific pressure, a zone of pressure, or that a maximum or minimum predetermined pressure level has been reached. The minimum or maximum pressure is predetermined and is dependent upon the specific airway device used and the patient on which the device is being used.

The pressure monitoring means is adapted to continually monitor at least one pressure within the inflatable sealing portion 10 of the airway device in one embodiment of the invention. The pressure monitoring means is adapted to intermittently monitor at least one pressure level within the inflatable sealing portion 10 of the airway device in another embodiment of the invention.

In a specific embodiment of the invention the inflatable airway device of the invention comprises at least one laryngeal airway comprising an inflatable sealing portion 10 and at least one balloon assembly and a pressure monitoring means comprising a gauge adapted to continuously measure at least one pressure level within the sealing portion 10 of the laryngeal airway.

In view of the above, it will be seen that all the objects and features of the present invention are achieved, and other advantageous results obtained. The description of the invention contained herein is illustrated only, and is not intended in a limiting sense.

What is claimed:

1. An artificial inflatable airway device comprising at least one inflatable sealing portion and a pressure monitoring means operably and integrally connected to the inflatable sealing portion, the pressure monitoring means adapted to measure at least one pressure within the sealing portion of the inflatable airway device.

2. The airway device of claim 1 wherein the inflatable sealing portion comprises at least one pressurized portion.

3. The airway device of claim 2 wherein the pressure monitoring means is attached to the pressurized portion of the airway device.

4. The airway device of claim 1 wherein the inflatable sealing portion is a balloon assembly.

5. The airway device of claim 4 wherein the pressure monitoring means lies within the balloon assembly.

6. The airway device of claim 1 wherein the pressure monitoring means comprises a gauge.

7. The airway device of claim 6 wherein the gauge shows at least one of a specific pressure, a zone of pressure, the zone of pressure corresponding to at least one of a pressure within the pressure monitoring means or a pressure within a sealing portion of the airway device, a minimum predetermined pressure and a maximum predetermined pressure.

8. The airway device of claim 1 wherein the airway device comprises an endotracheal tube.

9. The airway device of claim 1 wherein the airway device comprises a tracheal tube.

10. The airway device of claim 1 wherein the airway device comprises a laryngeal airway.

11. The airway device of claim 1 wherein the airway device comprises a supraglottic laryngeal airway.

12. The airway device of claim 1 wherein the airway device comprises an esophogeal obterator airway.

13. The airway device of claim 1 wherein the airway device comprises a pharyngeal airway.

14. The airway device of claim 1 wherein the pressure monitoring device is adapted to continually monitor at least one pressure within the inflatable sealing portion of the airway device.

15. The airway device of claim 1 wherein the pressure monitoring device is adapted to intermittently monitor at least one pressure within the inflatable sealing portion of the airway device.

16. The airway device of claim 1 wherein the pressure monitoring device is adapted to report the pressure within the sealing portion of the airway device to a user.

17. The airway of claim 1 wherein the pressure monitoring device is adapted to report to a user when the pressure within the sealing portion has reached a predetermined level.

18. An inflatable airway device comprising:
at least one laryngeal airway comprising an inflatable sealing portion and at least one balloon assembly; and
a pressure monitoring means integrally attached to the at least one laryngeal airway, the pressure monitoring means within the at least one balloon assembly and comprising a gauge adapted to continuously measure at least one pressure level within the sealing portion of the laryngeal airway,
wherein the pressure monitoring means is adapted to continuously report the pressure within the sealing portion of the laryngeal airway device.

19. An inflatable airway device comprising:
at least one laryngeal airway comprising an inflatable sealing portion and at least one balloon assembly; and
a pressure monitoring means integrally attached to the at least one laryngeal airway, the pressure monitoring means within the at least one balloon assembly and comprising a gauge adapted to intermittently measure at least one pressure level within the sealing portion of the laryngeal airway,
wherein the pressure monitoring means is adapted to intermittently report the pressure within the sealing portion of the laryngeal airway device.
